(19) United States
(12) Patent Application Publication
(10) Pub. No.: US 2005/0065616 A1
(43) Pub. Date: Mar. 24, 2005

(54) Implantable hydrogel with resorbable shell for use as an endoprothesis

(75) Inventors: Ieva Ankorina-Stark, Niva (DK); Boris Ivanovitch Pavlyk, Soborg (DK)

Correspondence Address: BIRCH STEWART KOLASCH & BIRCH, PO BOX 747, FALLS CHURCH, VA, 22040-0747, US

(73) Assignee: Contura SA, Montreux (CH)

(21) Appl. No.: 10/913,458

(22) Filed: Aug. 9, 2004

Related U.S. Application Data
(60) Provisional application No. 60/493,323, filed on Aug. 8, 2003.

(51) Int. Cl. A61F 2/02; A61F 2/12; A61F 2/28

(52) U.S. Cl. 623/23.75; 600/30; 623/8; 623/23.41

(57) ABSTRACT

An endoprosthesis comprised of a resorbable outer shell or coating material housing a filling material provides practical and cosmetic advantages. The outer shell or coating is resorbed back into the body within 12 months of implantation. Materials such as polyurethane, at an appropriate thickness, serve as an outer shell and provide the endoprosthesis with a shape as well as rendering the endoprosthesis implantable and easily handled by the surgeon. Once implanted, the outer shell or coated is resorbed into the body. The filling material, on the other hand, is biostable and biocompatible. The filling material is in the form of a hydrogel with a viscosity suited for the purpose. Polyacrylamide is a well suited hydrogel since it is biocompatible and can be formulated to the appropriate viscosity. The endoprosthesis of the invention can be used for soft-tissue replenishment such as in the face and breasts, or in the joints for support, or in an internal conduit to provide bulking.

### FIELD OF THE INVENTION

A biostable hydrogel housed in a resorbable polymeric unit provides for an implantable endoprosthesis use in the treatment of urinary incontinence, anal incontinence, vesicouretal reflux, reflux oesophagitis, mammoplasty, phalloplasty and soft-tissue filling such as for body contouring and as a facial endoprosthetic.

### GENERAL BACKGROUND

Natural and synthetic polymers such as collagen, soya, glycerol, silicone, polyacrylamide, polyvinylpyrolidone and hyaluronic acid have been utilised as endoprostheses. Materials used for endoprostheses generally try to imitate the natural soft tissue and are intended to be safe to the health of the patient.

Implantable endoprostheses typically comprise a filling material and outer shell. The outer shell functions to protect the body from the spreading of the filling material outside the targetted body area and/or to protect the body from the toxic effects of the filling material over time in the body and/or to provide the desired form and shape of the endoprosthesis.

The present inventors have conceived and developed an implantable endoprothesis wherein the outer shell is intended to be resorbed into the body,

U.S. Pat. No. 5,658,329 relates to an implantable endoprosthesis for soft tissue medical implants and addresses the use of a filling material in a shell wherein the filling material is a gel containing only biocompatible ingredients with a low total solids content, is excretable or metabolizable and sterile. This addressed the problem that in the event the shell ruptures, either as a result of accidents or through crease-fold flaw or failure, the filling material escapes into the body. For this reason, the filing material is judiciously selected. The present invention differs from U.S. Pat. No. 5,658,329 in that is the actual intention of the present invention for shell to degrade and expose the filling material to the body.

EP 0 784 987 discloses a prosthesis with an outer shell. The outer shell is preferably constructed from a flexible inflatable material. The filling material is a dehydrated polymer which is wetted to fully swell after implantation into the body. The invention according to EP 0 784 987 selects a material for an outer shell which suitably inflates as the dehydrated filling material swells upon wetting. Moreover, the invention according to EP 0 784 987 intends to avoid rupture of the shell, optionally be providing a lubricant between the filling material and the inner wall of the outer shell. Conversely, the present invention selects a material for an outer shell and defines the outer shell such that it degrades and is resorbed into the body.

Similarly, EP 0 727 232 discloses a prosthetic device wherein here shall be no degradation of shell or membrane physical properties. This opposes the intention of the present invention wherein instead the shell is designed to decompose and release the filling material.

EP 1 214 953 discloses a mammary prosthesis comprising polyacrylamide hydrogel in a rounded shell of medical high polymer elastic material, namely silicone.

Endoprostheses comprising outer shells provide the advantages implantability over injectable endoprostheses. However, outer shells also have numerous disadvantages such as seams, valves, hardening and rigidity and un-natural appearance. The present invention provides the advantages of implantation of the endoprosthesis, but due to the resorbability of the shell, avoids the disadvantages of outer shells.

### SUMMARY OF THE INVENTION

The invention relates, in a first aspect to a biostable hydrogel housed in a resorbable polymeric unit. Alternatively stated, the invention relates to an endoprosthesis comprising a biostable hydrogel housed in a resorbable polymeric unit (or outer shell). Furthermore, the invention also relates to the use of a biostable hydrogel and a resorbable polymeric unit for the preparation of an endoprosthesis for the treatment of facial deformities, mammoplasty, phalloplasty, urinary incontinence, anal incontinence, vesicouretal reflux or reflux oesophagitis, wherein the resorbable polymeric unit houses the hydrogel.

### DESCRIPTION OF THE INVENTION

A first aspect of the invention relates to a hydrogel comprising a resorbable outer unit, suitable for use as an implantable endoprosthesis, namely a biostable hydrogel housed in a resorbable polymeric unit. The invention relates to a composite device suitable for use as an implantable endoprosthesis comprising a resorbable outer unit housing a hydrogel.

The resorbable polymeric unit which serves to house the hydrogel may be an outer surface, an outer coating, outer layer, outer membrane, outer envelope, outer sleeve, outer casing, or outer shell to the hydrogel.

The resorbable polymeric unit may be enveloping the hydrogel or suitable for enveloping the hydrogel. The resorbable polymeric unit may be a mechanical/physical envelope to the hydrogel such as in the form of a distinct casing unit within which one can manually insert the hydrogel, thus forming a loose associating with the hydrogel. Alternatively the resorbable polymeric unit may be a chemical envelope to the hydrogel such as in the form of a film or coating unit which is applied to the surface of the gel or made by chemical modification of the surface of the hydrogel.

The term “resorbable” in the context of the term “polymeric unit” is intended to mean that the outer unit degrades within the body, namely that the outer unit is made of a material that degrades within the animal body. The term “resorbable” is intended to be essentially synonymous with the term bioabsorbable. The degraded material is transported away from the tissue area and eliminated from the general circulation.

The resorbable polymeric unit is an outer unit which serves to house the hydrogel. The resorbable polymeric unit provides a temporary barrier between the hydrogel and the surrounding tissue. The resorbable outer unit is a flexible material which houses the hydrogel. The resorbable outer unit further allows for the hydrogel to be implantable. The resorbable outer unit provides for a more easily handled device to the medical practitioner during the implantation of the gel compared to the gel without the presence of this resorbable outer unit. The practitioner can apply mechanical force during the implantation.

Materials suitable for use as the resorbable polymeric unit include polymers such as polyurethanes, aliphatic polyesters, poly(amino acids), copoly(ether-esters), polyalkylene oxalates, polyamides, poly(glycolic acid),polylactide, tyrosine derived polycarbonates, poly(iminocarbonates), polyorthoesters, polyoxaesters, polyamidoesters, polyoxaesters containing amine groups, poly(anhydrides), polyphosphazenes, collagen, elastin, bioabsorbable starches, and combinations and copolymers thereof.

The resorbable polymeric unit which houses the hydrogel is also referred to as the outer shell. The material of the outer shell is selected so that it substantially or fully degrades within 12 months, such as from 9 to 12 month, such as from 6 to 12 months. That is to say that within 12 months, at least 80% of the polymeric material of the outer shell has degraded, such as at least 85%, preferably at least 90%, more preferably at least 95%. Typically, at least 80% of the polymeric material has degraded within 10 months, such as within 9 months, such as within 6 to 9 months, such as 4, 5, 6, 7, 8 or 9 months.

Optionally, the outer shell may be coated with a chemical or enzyme which accelerates the degradation of the polymeric material of the out shell. In some embodiments, in may be preferable that the outer shell is coated with a chemical or enzyme which retards the degradation of the polymeric material.

The thickness of the outer shell is typically such that the polymeric material of the outer shell is substantially or fully degraded within 9 months. The thickness of the outer shell which allows for the desired degradation within 9 months will obviously depend on the selection of the polymeric material. Typically, the outer shell has a thickness of between 0.1 μm to 20 mm, such as 0.1 μm to 18 mm, more typically 0.1 μm to 15 mm, preferably 0.1 μm to 10 mm, more preferably 0.1 μm to 8 mm, such as 0.1 μm to 6 mm, such as 0.1 μm to 5 mm, suitably 0.1 μm to 4 mm, such as 0.1 μm to 3 mm, more suitably selected from 0.1 μm to 2 mm, 0.1 μm to 1 mm and 1 μm to 0.5 mm. In another embodiment, the outer shell typically has a thickness of 0.5 μm to 15 mm, preferably 1 μm to 10 mm, more preferably 1 μm to 8 mm, such as 1 μm to 6 mm, such as 1 μm to 5 mm, suitably 1 μm to 4 mm, such as 1 μm to 3 mm, more suitably selected from 1 μm to 2 mm, 1 μm to 1 mm and 1 μm to 0.5 mm. Alternatively, the outer shell may typically have a thickness of 5 μm to 15 mm, preferably 5 μm to 10 mm, more preferably 5 μm to 8 mm, such as 5 μm to 6 mm, such as 5 μm to 5 mm, suitably 5 μm to 4 mm, such as 5 μm to 3 mm, more suitably selected from 5 μm to 2 mm, 5 μm to 1 mm and 5 μm to 0.5 mm. Furthermore, the outer shell may typically have a thickness of 10 μm to 15 mm, preferably 10 μm to 10 mm, more preferably 10 μm to 8 mm, such as 10 μm to 6 mm, such as 10 μm to 5 mm, suitably 10 μm to 4 mm, such as 10 μm to 3 mm, more suitably selected from 10 μm to 2 mm, 10 μm to 1 mm and 10 μm to 0.5 mm.

In a most preferred embodiment the outer shell has a thickness of 0.1 mm to 2.5 mm, such as 0.1 to 2 mm, such as 0.25 to 1.5 mm.

In a preferred embodiment, the resorbable polymeric unit is polyurethane or a co-polymer thereof, such as a block co-polymer. In a preferred embodiment, the resorbable outer unit comprises at least 80% polyurethane, such as at least 90% polyurethane, such as 95% polyurethane, such as 99% or 100%.

The polyurethane may be linear, branched or cross-linked, preferably linear. The tensile strength of outer shells comprising at least 80% polyurethane was shown to dramatically decrease after implantation.

In a preferred embodiment, the resorbable polymeric unit is made of a material which, as it decomposes, is bio-absorbed or resorbed, is not injurious to either the hydrogel or the tissue surrounding the endoprosthesis.

The resorbable outer unit houses a hydrogel which serves as the filling material. The hydrogel is a polymer swollen in a liquid, said liquid not being a solvent to the polymer. In a typical embodiment, the liquid is water or an aqueous solution such as a saline solution. The filling material is most preferably comprised of non-resorbable material.

In suitable embodiments of the invention, the solid weight content of the hydrogel is 0.1 to 25%, such as 0.5-20%, such as 1-10%, preferably 1-7% , more preferably 1-5%, even more preferably 1-4%. One advantage of the providing a housing unit to the hydrogel is the ability to use hydrogels of lower viscosity. The hydrogel may thus have a lower solid weight content than conventional hydrogels such as 0.1-10%, preferably 0.1-7%, more preferably 0.5-5%, such as 0.5 to 4.5%, even more preferably 0.5-4%, such as 0.5-3.5%, such as 0.75 to 3.5%, most preferably 1-3.5% or 1-3.25%.

The hydrogel housed by the resorbable polymeric unit is most suitably a cross-linked polymer comprising polyacrylamide or a co-polymer thereof. In a preferred embodiment, at least 80% of the polymeric content of the hydrogel is polyacrylamide, such as at least 90%, more preferably at least 95%, even more preferably at least 99%, such as 100% polyacrylamide.

In a most preferred embodiment, the hydrogel is 0.5 to 25% polyacrylamide. In a combination of preferred embodiments of the invention, the polymeric material of the filling material typically comprises at least 90% polyacrylamide with a solid weight content of the hydrogel is 0.1 to 25%, such as 0.5-20%, such as 1-10%, preferably 1-7% , more preferably 1-5%, even more preferably 1-4%. The polymeric material of the filling mateial may alternatively comprise at least 95% polyacrylamide with a solid weight content of the hydrogel of 0.1-10%, preferably 0.1-7%, more preferably 0.5-5%, such as 0.5 to 4.5%, even more preferably 0.5-4%, such as 0.5-3.5%, such as 0.75 to 3.5%, most preferably 1-3.5% or 1-3.25%.

Together, the hydrogel housed in a resorbable polymeric unit is termed herein as the composite product of the hydrogel and the outer polymeric unit. The composite product is intended for use as an implantable endoprosthesis.

In a preferred aspect of the invention, the composite material product for use as an implantable endoprosthesis comprises a resorbable polymeric unit of consisting of polyurethane or co-polymer thereof, and a hydrogel of a polymer wherein at least 90% of the polymeric content is polyacrylamide and the hydrogel has a solid weight content of 0.5 to 25% and the hydrogel comprises is water or an aqueous solution.

In a further preferred embodiment of the invention, the hydrogel has a solid weight content of 0.5 to 25% polymeric material, wherein the polymeric material a polymeric content of at least 90% polyacrylamide, and the resorbable polymeric unit comprises essentially of polyurethanes and copolymers thereof.

In a suitable embodiment, the hydrogel may be very fluid such that they may be characterised in that it has a complex viscosity not less than 1 Pas, such as not less than 2 Pas, such as not less than 3, 4 or 5 Pas. In a suitable embodiment, the hydrogel comprising may have a viscosity of 1 to 90, such as 2 to 80 Pas, preferably from about 2 to 75, such as from about 5 to 60, 6 to 40, 6 to 20, such as 6 to 15 Pas.

As stated, an advantage of using a housing unit over an injectable material is the ability to use filling materials of lower viscosity. Thus, in one embodiment of the invention, the hydrogel has a viscosity of 1 to 20 Pas, such as 2 to 20 Pas, such as 5 to 20 Pas, such as 2 to 15 Pas.

Conversely, an advantage of using a housing unit over an injectable material is the ability to use filling materials of higher viscosity. Thus, in an alternate set of embodiments, the hydrogel has a viscosity of 5 to 100 Pas, such as 10 to 90 Pas, preferably from 15 to 80 Pas, such as 20 to 60 Pas.

The hydrogel may be characterised in that it has elasticity module of not less than 10 Pa, such as not less than 20, 25, 30, 31, 32, 33, 34 or 35 Pa, such as not less than 38 Pa. Typically, the hydrogel has an elasticity module from about 10 to 700 Pa, such as about 35 to 480 Pa.

These rheological features are in due in part to the degree of cross-linking and to the degree of swelling of the hydrogel. The hydrogel comprising polyacrylamide may be characterised in that the cross-linked polyacrylamide is to such as degree so as to have an efficient cross-linking density of about 0.2 to 0.5%, preferably about 0.25 to 0.4%.

The cross-linking density is in turn due in part to the molar ratio between the acrylamide and the cross-linking agent. In a preferred embodiment, the cross-linking agent is methylene-bis-acrylamide. Typically the ratio between acrylamide and the cross-linking agent is in the range 175:1 to 800:1, such as from 225:1 to 600:1, preferably from 250:1 to 550:1, most preferably from 250:1 to 500:1. The absolute and relative amount of the redox agent used in the preparation of the polyacrylamide may be TEMED. The redox agent and the initiator also influence the degree of cross-linking.

As stated, the composite device is intended for use as an endoprosthesis. In a suitable embodiment, the resorbable polymeric unit is a distinct casing unit which forms a removable shell or envelope to the hydrogel. The hydrogel can be provided in assembled form or for assembly. One aspect of the invention relates to the resorbable polymeric unit which houses a hydrogel for use as an implantable endoprosthesis. Moreover, a suitable embodiment of the invention relates to a kit comprising a biostable hydrogel and a resorbable polymeric unit, in any form as defined supra.

In a combination of embodiments, the hydrogel has a 0.5 to 10% solid weight content, the hydrogel comprising at least 95% polyacrylamide in terms of polymeric content, the hydrogel having a viscosity of 15 to 80 Pas; and wherein the resorbable polymeric unit or outer shell which houses the hydrogel is polyurethane or a co-polymer thereof comprising at least 90% polyurethane, the outer shell having a thickness of 0.1 μm to 10 mm.

In a combination of embodiments, the hydrogel has a 0.5 to 10% solid weight content, the hydrogel comprising at least 95% polyacrylamide in terms of polymeric content, the hydrogel having a viscosity of 1 to 15 Pas; and wherein the resorbable polymeric unit or outer shell which houses the hydrogel is polyurethane or a co-polymer thereof comprising at least 90% polyurethane, the outer shell having a thickness of 0.1 μm to 10 mm.

In a combination of embodiments, the hydrogel has a 0.5 to 3.5% solid weight content, the hydrogel comprising at least 95% polyacrylamide in terms of polymeric content, the hydrogel having a viscosity of 1 to 15 Pas; and wherein the resorbable polymeric unit or outer shell which houses the hydrogel is polyurethane or a co-polymer thereof comprising at least 90% polyurethane, the outer shell having a thickness of 0.1 μm to 10 mm.

In a combination of embodiments, the hydrogel has a 0.5 to 10% solid weight content, the hydrogel comprising at least 95% polyacrylamide in terms of polymeric content, the hydrogel having a viscosity of 15 to 80 Pas; and wherein the resorbable polymeric unit or outer shell which houses the hydrogel is polyurethane or a co-polymer thereof comprising at least 90% polyurethane, the outer shell having a thickness of 0.1 μm to 10 mm.

In a combination of embodiments, at least 80% of the resorbable polymeric unit is degraded within 6 months of its implantation; and the hydrogel having a 0.5 to 3.5% solid weight content, the hydrogel comprising at least 95% polyacrylamide in terms of polymeric content, the hydrogel having a viscosity of 1 to 15 Pas.

In a combination of embodiments, at least 80% of the resorbable polymeric unit is degraded within 6 months after its implantation; and the hydrogel has a 1 to 10% solid weight content, the hydrogel comprising at least 95% polyacrylamide in terms of polymeric content, the hydrogel having a viscosity of 15 to 80 Pas.

The device as defined herein is intended, in one embodiment, for use as an implantable endoprosthesis in the form of soft-tissue filler or soft-tissue augmentation prosthesis, such as in the soft-tissue of the body (body contouring) or for the face, as a facial endoprosthetic. Thus, the device may be shaped and sized according to its intended use. The device may be used for reshaping or reforming a persons nose, lip, cheek or other facial contours. The endoprosthesis may be customized in terms of shape to conform to a particular scar or deformity.

The device for use as an implantable endoprosthesis may be used in mammoplasty or phalloplasty. It may be for use in reshaping a person's buttock.

The device as defined herein may also be used as in a similar manner as bulking agents are used in the treatment of urinary incontinence, anal incontinence, vesicouretal reflux or reflux oesophagitis. The device may in the form of a ball which one implants in the submucoal layer in the appropriate conduit. Alternatively, the device may be in the form of a ring or bulbous ring which is similarly implanted to provide resistance in the appropriate conduit.

Similarly, the device as defined herein may be used in joints in the human or animal body.

The invention further relates to the use of a resorbable polymeric unit as an outer shell for the preparation an endoprosthesis, said outer shell housing a polymeric hydrogel, for the treatment of facial deformities, mammoplasty, phalloplasty, of urinary incontinence, anal incontinence, vesicouretal reflux or reflux oesophagitis. The hydrogel preferably comprises as polymeric material, at least 80% polyacrylamide. The endoprosthesis is as defined herein. Similarly, the invention relates to the use of a biostable hydrogel and a resorbable polymeric unit for the preparation of an endoprosthesis for soft-tissue filling of human, such as for soft-tissue filling of the body (body contouring) or for the face, wherein the resorbable polymeric unit houses the hydrogel. Furthermore, the invention also relates to the use of a biostable hydrogel and a resorbable polymeric unit for the preparation of an endoprosthesis for the treatment of facial deformities, mammoplasty, phalloplasty, urinary incontinence, anal incontinence, vesicouretal reflux or reflux oesophagitis, wherein the resorbable polymeric unit houses the hydrogel. The endoprosthesis of the invention is suitable for the bulking of conduits in mammals suffering from urinary incontinence, anal incontinence, vesicouretal reflux or reflux oesophagitis. Thus, in a further embodiment, the device of the invention is a bulking agent.

In operative procedures involving mammoplasty, the endoprosthesis of the invention showed good cosmetic effect, as well as biocompatibility in terms of the polyurethane and its degradation product and in terms of a polyacrylamide hydrogel itself. Studies demonstrated the formation of connective tissue and in the timely degradation of the polymeric material forming the outer shell.

1. A biostable hydrogel housed in a resorbable polymeric unit.

2. The hydrogel according to claim 1, wherein the resorbable polymeric unit is an outer surface, outer coating, outer layer, outer membrane, outer envelope, outer sleeve, outer casing, or outer shell to the hydrogel.

3. The hydrogel according to any one of the preceding claims wherein the resorbable polymeric unit is a film or coating applied to the surface of the hydrogel or made by chemical modification of the surface of the hydrogel.

4. The hydrogel according to claim 1, wherein the resorbable polymeric unit is a distinct casing unit which forms a removable shell or envelope to the hydrogel.

5. The hydrogel according to claim 1, wherein the resorbable polymeric unit consists of polymers selected from the group consisting of polyurethanes, aliphatic polyesters, poly(amino acids), copoly(ether-esters), polyalkylene oxalates, polyamides, poly(glycolic acid),polylactide, tyrosine derived polycarbonates, poly(iminocarbonates), polyorthoesters, polyoxaesters, polyamidoesters, polyoxaesters containing amine groups, poly(anhydrides), polyphosphazenes, collagen, elastin, bioabsorbable starches, and combinations and copolymers thereof.

6. The hydrogel according to claim 1, wherein the resorbable polymeric unit consists of polymers selected from the group consisting of polyurethanes and copolymers thereof.

7. The hydrogel according to claim 1, wherein the resorbable polymeric unit comprises at least 80% polyurethane, such as at least 90% polyurethane, such as 95% polyurethane, such as 99% or 100%.

8. The hydrogel according to claim 1, wherein the hydrogel has a solid weight content of 0.1 to 25% polymeric material, such as 0.5-20%, such as 1-10%, preferably 1-7%, more preferably 1-5%, even more preferably 1-4%.

9. The hydrogel according to claim 1, wherein the hydrogel is cross-linked polymer comprising polyacrylamide or a co-polymer thereof.

10. The hydrogel according to claim 1, wherein the hydrogel is cross-linked polymer with a polymeric content of at least 80% polyacrylamide, such as at least 90%, more preferably at least 95%, even more preferably at least 99%, such as 100% polyacrylamide.

11. The hydrogel according to claim 1, wherein the hydrogel has a solid weight content of 0.5 to 25% polymeric material, wherein the polymeric material a polymeric content of at least 90% polyacrylamide, and wherein the resorbable polymeric unit consists of polymers selected from the group consisting of polyurethanes and copolymers thereof.

12. The hydrogel according to claim 1, wherein the resorbable polymeric unit comprises a polymeric material such that at least 80% of the polymeric unit is substantially or fully degraded within 12 months after implantantion.

13. A composite product suitable for use as an endoprosthesis comprising a biostable hydrogel housed in a resorbable polymeric unit as defined in claim 1.

14. A kit comprising a biostable hydrogel and a resorbable polymeric unit.

15. Use of a biostable hydrogel and a resorbable polymeric unit for the preparation of an endoprosthesis for soft-tissue filling of human, such as for soft-tissue filling of the body (body contouring) or for the face, wherein the resorbable polymeric unit houses the hydrogel.

16. Use of a biostable hydrogel and a resorbable polymeric unit for the preparation of an endoprosthesis for the treatment of urinary incontinence, anal incontinence, vesicouretal reflux or reflux oesophagitis, wherein the resorbable polymeric unit houses the hydrogel.
